(19) United States
(12) Patent Application Publication
(10) Pub. No.: US 2023/0210757 A1
(43) Pub. Date: Jul. 6, 2023

(54) IMPROVEMENTS IN OR RELATING TO ORGANIC COMPOUNDS

(71) Applicant: Givaudan SA, VERNIER (CH)

(72) Inventors: Anne HUMEAU, Trégastel (FR); Marie MEUNIER, Les Petites Loges (FR); Romain REYNAUD, Toulouse (FR); Amandine SCANDOLERA, Reims (FR)

(21) Appl. No.: 18/000,526

(22) PCT Filed: Jun. 15, 2021
(86) PCT No.: PCT/EP2021/066062

§ 371 Date: Dec. 2, 2022

(30) Foreign Application Priority Data
Jun. 16, 2020 (GB) 2009109.6

(51) Int. Cl. A61K 8/9711; A61Q 19/02

(52) U.S. Cl. CPC A61K 8/9711; A61Q 19/02

(57) ABSTRACT

An extract of Himanthalia elongata and a cosmetic composition comprising a carrier and said extract is provided. The extract and the composition show whitening and anti-ageing activities.

The present invention is directed to a Himanthalia elongata extract and its use for skin treatment. It is further directed to cosmetic compositions comprising a Himanthalia elongata extract.

Himanthalia elongata is an edible brown alga, also known as sea spaghetti. It is a biannual alga with a two-step morphology: a vegetative form and a reproductive form. The reproductive form has long thongs which look like hair.

H. elongata is found in the Baltic Sea, the North Sea and the north east Atlantic Ocean from Scandinavia south to Portugal. For example, this alga can be harvested in the north of Brittany, France, for example near Roscoff or near Landrellec, preferably in the area between Ile Grande and Pleubian, between May and August (but preferentially in July or August).

Himanthalia elongata contains many original molecules that might be of interest for dermocosmetics. For example, this brown seaweed contains two polyols. The first one, D-mannitol is common to brown algae, but the second, D-altritol or D-talitol was discovered for the first time in nature in Himanthalia elongata and is considered as a rare sugar. This polyol is involved in intracellular osmotic regulation, in the energy storage and in the protection of biological macromolecules. Himanthalia elongata is also rich in peptides and amino-acids and contains polysaccharides (alginates, laminarines, fucans) and polyphenols.

It was surprisingly found that Himanthalia elongata extract, and especially particular fractions thereof, can have impressive skin care properties.

In a first aspect, the present invention provides a Himanthalia elongata extract. Said extract is useful for compositions and methods for skin treatment. In particular, they are cosmetic, non-therapeutic compositions and methods.

The Himanthalia elongata extract is an aqueous extract, obtained by treatment of the frozen algae under acidic conditions. In particular, the extract is obtained by treatment with aqueous acid.

The Himanthalia elongata extract of the invention is an enriched extract and contains polyphenols and in particular phlorotannins. Phlorotannins are a group of complex polymers of phloroglucinol (1,3,5-trihydroxybenzene) unique to macroalgae. These phenolic compounds are integral structural components of the cell wall in brown algae, but also play many secondary ecological roles such as protection from UV radiation and defense against grazing. The extract comprises fatty acids, citric acid, D-mannitol, D-altritol, and different phlorotannins in the form of oligomers and phloroglucinol polymers with various polymerization degrees (DP).

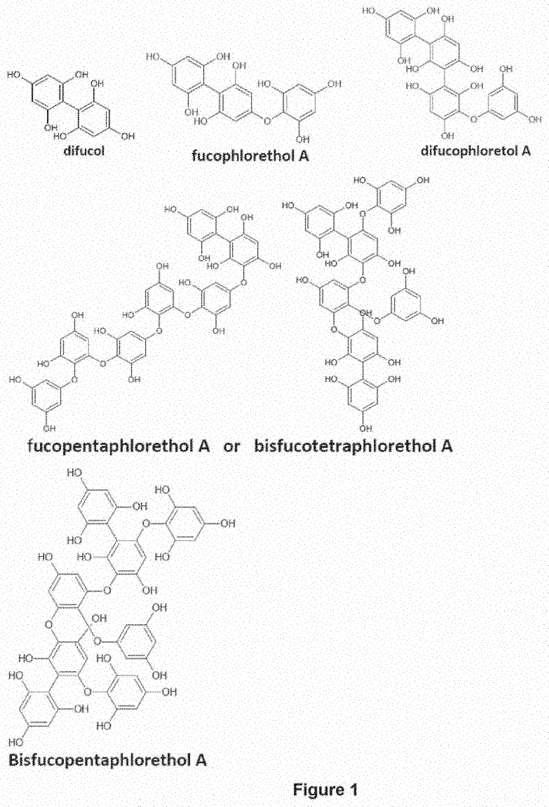

Some examples of phlorotannins present in Himanthalia elongata are shown in FIG. 1.

In a first in tubo screening of Himanthalia elongata extract, anti-tyrosinase, anti-hyaluronidase, anti-collagenase and anti-elastase activities have been observed (examples 3-6), which are of interest in the field of skin care.

The anti-tyrosinase activity is associated with skin whitening which is looked for in cosmetics. The Himanthalia elongata extract is able to interact with tyrosinase, an enzyme for controlling the production of melanin.

On contact with its natural substrate, the enzyme tyrosinase catalyzes the oxidation of L-DOPA to the reactive intermediate dopaquinone, which reacts further, eventually leading to melanin oligomers. The presence of inhibitors of this enzyme makes it possible to reduce the rate of dopaquinone formation.

The anti-hyaluronidase, anti-collagenase and anti-elastase activities of Himanthalia elongata extract are associated with anti-aging properties like for example reduction of fine lines and wrinkles, increase of skin elasticity, firmness and hydration, and others which are also looked for in cosmetics and skin care.

Hyaluronic acid is a compound naturally produced in the dermis of the skin, and is continuously degraded by an enzyme called hyaluronidase. Thus, with age, the ratio between hyaluronic acid synthesized by the cells and hyaluronic acid degraded by hyaluronidase decreases. This causes a reduction in dermal moisturization and progressive sag, which leads to the appearance of wrinkles. Inhibition of hyaluronidase (anti-hyaluronidase activity) would permit to reduce the amount of degraded hyaluronic acid in the dermis and thus to limit the appearance of wrinkles.

The collagenase enzyme is capable of degrading molecules such as elastin, fibronectin, and collagen. These proteins present in the dermis, have an essential role in the extracellular matrix (ECM), and their degradation leads to premature aging of the skin. Therefore, agents that inhibit collagenase activity (show anti-collagenase activity) may have beneficial effects for maintaining healthy skin by preventing degradation of the dermal matrix. Collagenase is capable of cleaving “X-gly” bond of synthetic peptides, whose amino terminal is blocked.

Elastin is a dermal protein which is a constituent of the extracellular matrix (ECM), and it is mainly contributing to skin firmness and elasticity. Over time, the metabolism of the ECM proteins slows down. In parallel, enzymatic activity increases, including that of elastase, which breaks down elastin. One way to prevent such a loss of elasticity is to use active ingredients with anti-collagenase activities that are able to inhibit these enzymes.

Therefore, the observed anti-tyrosinase, anti-hyaluronidase, anti-collagenase and anti-elastase activities of Himanthalia elongata extract suggest whitening and anti-aging properties of the extract. It is concluded, that a cosmetic composition comprising an extract of Himanthalia elongata is able to reduce fine lines and wrinkles, and to increase skin elasticity, firmness and hydration. It is also concluded that a cosmetic composition comprising an extract of Himanthalia elongata is able to whiten and/or lighten the skin.

A comparison of Himanthalia elongata extract with phenylethyl resorcinol (which is a whitening compound available on the market) has shown comparable or even better whitening effects. In addition, Himanthalia elongata extract shows anti-aging properties. Moreover, Himanthalia elongata extract is a natural product, obtainable from renewable sources.

The whitening activity of Himanthalia elongata extract was investigated in more detail.

First, melanin synthesis inhibition by Himanthalia elongata extract on normal human melanocytes was observed, confirming the whitening activity at in vitro level.

In tubo testing evidenced that Himanthalia elongata extract has a strong anti-tyrosinase activity. Therefore, it could be very efficient on hyper pigmented disorder such as pigmented ageing spots. Indeed, the tyrosinase is a key enzyme involved in melanin biogenesis which is responsible for skin colour. An overactivity of this enzyme leads to dermatological disorders among them ageing spots that could be divided two types named senile and solar lentigo.

So in a next step, ex vivo testing on ageing spots regarding Senile lentigo (example 7) and Solar lentigo (example 8) was performed. It was found that Himanthalia elongata extract is able to reduce the melanin content, to reduce oxidized proteins, to control the inflammation related to solar lentigo and to improve the skin barrier.

The senile lentigo is related to an increase of cell senescence and especially dermal fibroblasts closely based all along the dermo-epidermal junction. They play an active role on the control of skin pigmentation due the SDF-1 (stromal cell-derived factor 1) release. Unfortunately with ageing a drastic reduction of SDF-1 expression is observed, leading to hyperpigmentation and pigmented ageing spots formation.

The solar lentigo is mediated by an UV exposure which leads to cellular senescence and so surprisingly to a subsequent decrease of SDF-1 expression related to a loss of pigmentation control. Furthermore, UV exposure also leads to an increase of oxidized proteins due to a reduction of proteasome activity correlated to an increase of stratum corneum thickness. As a consequence, a strong increase of lipofuscin is observed, which is well-known to be involved in ageing spots formation. In parallel, an increase of UV-induced inflammation associated with an over stimulation of melanogenesis is observed. All these disrupted mechanisms lead to a hyperpigmentation and pigmented ageing spots formation.

SDF-1 has been described in the literature to be associated to dermis' fibroblasts senescence. Indeed, in senescence condition, these fibroblasts lose their capacity to express SDF-1 (Yoon et al. 2018). On another side, UV radiations are known to induce cellular senescence in fibroblasts and keratinocytes from the skin (Debacq-Chainiaux et al, 2012; Amaro-Ortiz et al, 2014). Thus, it was postulated that UV radiations which are involved in the formation of solar lentigo could also induce skin senescence. In such a case, an impact of UV radiations on SDF-1 expression in the papillary dermis would be expected, although not described in literature.

Surprisingly, it has been shown that SDF-1 is involved in hyperpigmentation related to UV exposure leading to solar lentigo. It was further shown, that this effect can be influenced by treatment with Himanthalia elongata extract.

Himanthalia elongata extract was found to have impact on senile and solar lentigo (see examples 7 and 8). It can be concluded, that a cosmetic composition comprising an extract of Himanthalia elongata is able to increase of SDF1 expression, thereby activating the pigmentation control. It can also be concluded that a cosmetic composition comprising an extract of Himanthalia elongata is able to reduce melanin biogenesis as observed by the reduction of melanin content.

Afterwards, an ex vivo study was performed on skin explants from different ethnic groups, including caucasian, asian and african origin (example 9). It was shown, that Himanthalia elongata extract is having a whitening activity causing an increase of unpigmented cells and a decrease of very pigmented cells. Himanthalia elongata extract has whitening efficacy on skin of various ethnicities. The effect is comparable or even better than that of phenylethyl resorcinol.

Finally, the whitening activity of Himanthalia elongata extract was tested in clinical studies. In a first trial, the reduction of pigmented spots on hands of Caucasian volunteers has been investigated (example 10). In a second trial, the reduction of pigmented spots on face of Asian volunteers has been studied (example 11). In a third trial, the reduction of pigmented spots on face of African volunteers has been investigated (example 12) Overall, it was demonstrated that Himanthalia elongata extract can reduce pigmented spots on skin, in particular on hands and face.

It was demonstrated that by application of the Himanthalia elongata extract on skin one or more of the following effects can be achieved: inhibition of tyrosinase, inhibition of hyaluronidase, inhibition of elastase, inhibition of collagenase, increase of SDF1 expression, reduction of melanin biogenesis, reduction of melanin content, reduction of oxidized proteins after UV exposure, reduction of lipofuscin bodies accumulation after UV exposure, reduction of inflammation after UV exposure, reduction of Stratum corneum thickness after UV exposure.

It was demonstrated that by application of the Himanthalia elongata extract on skin one or more of the following effects can be observed: skin whitening, activation of the pigmentation control, reduction of pigmentation or hyperpigmentation, reduction of skin redness, reduction of visible spots, reduction of ageing spots, reduction of skin ageing.

In a further aspect, the present invention provides a cosmetic composition comprising a carrier and Himanthalia elongata extract as a first active cosmetic ingredient. The Himanthalia elongata extract should be present in an amount of 2-5% by weight, preferably 2.5-3.5% by weight, more preferably 3% by weight of the cosmetic composition. The cosmetic composition can comprise further active cosmetic ingredients. The carrier in a cosmetic composition should be a dermatologically acceptable carrier.

In a further aspect of the present invention there is provided a cosmetic composition which is a skin care composition, Typical skin care compositions are for example creams, serums, essences, masks or lotions.

In a further aspect of the present invention there is provided a method of skin treatment, comprising the step of applying a cosmetic composition comprising an extract of Himanthalia elongata to the skin. In particular, the applied cosmetic composition is a skin cam composition.

In a further aspect of the present invention there is provided a method of skin treatment for skin whitening. Furthermore, the provided method of skin treatment is for anti-aging treatment.

In a further aspect of the present invention there is provided the use of Himanthalia elongata extract in skin care.

In a further aspect, the invention relates to a method of preparing an active cosmetic ingredient, comprising the step of extracting Himanthalia elongata.

For the extraction of Himanthalia elongata, the frozen alga is grinded and suspended in water, heated and treated with acid at elevated temperature. After the acidic hydrolysis, the reaction mixture is cooled, and the pH is set to 2.2. The solution is centrifuged and filtered to give the first extract. This extract is then purified by adsorption on resin, washing with water and desorption with ethanol. The ethanol is then evaporated, and water is added. The pH is adjusted to 3 before filtration at 0.2 μm. At the end of the filtration, glycerin is added. The obtained extract contains 1-30 g/L of polyphenols (quantified as anhydrous gallic acid equivalents), preferably 3-25 g/L of polyphenols, more preferably 5-20 g/L of polyphenols, even more preferably between 9-18 g/L of polyphenols.

The present invention is now further illustrated by means of the following non-limiting examples:

General: All raw data from in vitro to in vivo experiments were analyzed by Shapiro-Wilk test in order to determine if the data followed Gaussian law or not. If yes, data were analyzed using parametric paired or unpaired t student tests. If no, data were analyzed using non-parametric Wilcoxon test or non-parametric Mann-Whitney test A result is considered as significant if p<0.1 #, p<0.05*, p<0.01** and p<0.001**.

### EXAMPLE 1: PREPARATION OF HIMANTHALIA ELONGATA EXTRACT (HEX)

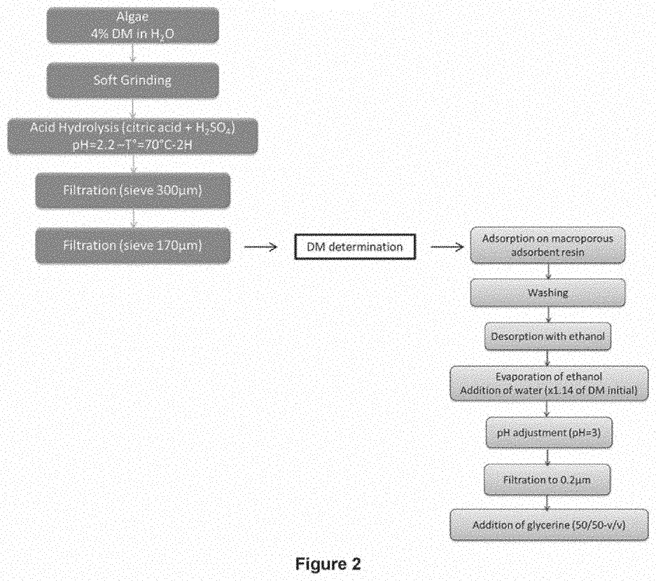

FIG. 2 shows a flow chart of the extraction process, carried out in two steps a) and b).

a) Unpurified Extract

A suspension of 4% of dry matter of Himanthalia elongata (harvested in the north of Brittany) in water was prepared. The seaweed was first cut into small pieces, macerated under stirring for 10 minutes, and then coarsely ground for 3 minutes with an ultra-turrax (12 000 rpm). The solution was then stirred and heated. When the temperature reached 65° C., citric acid was added (the amount of citric acid added corresponds to 7.5% of the dry matter of the suspension). Then, 1N sulfuric acid was added to set a pH=2.2. The acid hydrolysis lasted 2 h at 70° C. After 2 h, the solution was cooled, and filtered via a sieve of 170 μm. The unpurified extract could be centrifuged (12000 g, 18° C., 15 minutes) before contact with macroporous adsorbent resin (e. g. Diaion HP20).

b) Purified Extract (HEX)

The unpurified extract (about 4000 g) is put in contact with resin (about 1600 g, wet form) in a batch under stirring for 1 h After adsorption phase, the resin is washed three times for 5 minutes with water (3×4.6 L) to remove salts and polar compounds (e.g. sugars and proteins). Then chemicals adsorbed on resin are desorbed with ethanol 90% (maximum density 0.835, 1×4.6 L and 3×3.8 L for 30 minutes). Finally a new wash is performed with 3 L water for 5 minutes.

Ethanol fractions and final water eluate are pooled to be evaporated under vacuum rotary evaporator until ethanol content becomes lower that 1% and the mass of the concentrated solution is 1/85 of the masse of the initial extract. The pH is adjusted to 3.0 with sodium hydroxide and the product is filtered to 0.2 μm. The final product (purified extract) is obtained by diluting twice in glycerol (w/w). It contains between 9-18 g/L of polyphenols (quantified as anhydrous gallic acid equivalents.)

### EXAMPLE 2: ANALYSIS OF PHLOROTANNINS PRESENT IN THE HIMANTHALIA ELONGATA EXTRACT

a) Concentration of Phlorotannins

The aqueous extract of Himanthalia elongata (the purified extract obtained in Example 1) was diluted with distilled water and mixed with o-heptane+acetonitrile+methyl-tert-butyl ether in a separatory funnel. After mixing and decantation, the three resulting immiscible liquid phases (the upper phase 1 UP1, the middle phase MP1, the lower phase LP1) were separated.

The lower phase LP1 was diluted with distilled water and mixed with acetonitrile+methyl-ter-butyl ether. The two resulting liquid phases (the upper phase UP2 and the lower phase LP2) were separated. The upper phase UP2 was finally mixed with MP1. The solvent was evaporated under vacuum, resulting in a fraction enriched in phlorotannins, noted E-Phlor.

b) Fractionation

The extract E-Phlor, enriched in phlorotannins, has been fractionated by centrifugal partition chromatography (CPC) on a FCPE3000 instrument by Rousselet-Robatel-Kromaton, under the following conditions:
Injected mass 1.5 g of E-Phlor
Two-phase solvent system: methyl-tert-butyl ether/acetonitrile/water (4/1/5, v/v)
Stationary phase: lower phase of the two-phase solvent system (ascending mode)
Mobile phase: upper phase of the two-phase solvent system
Column Rotation speed: 1200 rpm
Flow rate of 20 mL/min

The mobile phase was pumped through the stationary phase for 60 minutes. During this elution step, the organic compounds having an affinity for the mobile phase were collected at outlet of the column. Then, the stationary phase was extruded. During this extrusion step, all the compounds remaining trapped in the column were recovered.

c) Fraction Analysis

Eleven successive CPC fractions F01 to F11 were obtained. All fractions were analyzed by ¹³C NMR on a Bruker Avance AVIII-600 spectrometer (Karlsruhe, Germany) equipped with a cryoprobe. All CPC fractions contained significant amounts of phlorotannins, as summarized in table 1, wherein * means presence in lower quantity and ** means presence in higher quantity of the phlorotannin with the indicated polymerization degree. LC/MS analyses were also performed to tentatively discriminate the various chemical structures of phlorotannins contained in the CPC fractions

TABLE 1
Distribution of phlorotannins in the CPC fractions.

| | | | PHLOROTANNIN DISTRIBUTION | | | | | | | | | | |
|---|---|---|---|---|---|---|---|---|---|---|---|---|---|
| | | | 6.1 | 6.4 | 6.6 | 7.4 | 7.5 | 8.1 | 8.3 | 8.5 | 8.6 | 9.3 | 9.4 |
| | | % | min | min | min | min | min | min | min | min | min | min | min |
| CPC | Mass | [symbol] | DP | DP | DP | DP | DP | DP | DP | DP | DP | DP | DP |
| Fractions | (mg) | extract | 2 | 4 | 3 | 3 | 3 | 4 | 6 | 4 | 7 | 7 | 5 |
| 01 | [symbol] | [symbol] % | [symbol] | | | | | | | | | | [symbol] |
| 02 | 183 | 11. [symbol] % | [symbol] | | | [symbol] | | [symbol] | | [symbol] | [symbol] | | [symbol] |
| 03 | [symbol] 8 | [symbol] % | [symbol] | | | [symbol] | [symbol] | [symbol] | | [symbol] | [symbol] | | |
| 04 | 30 | 2.0% | [symbol] | | | | [symbol] | [symbol] | | | | | [symbol] |
| 05 | 49 | [symbol] % | [symbol] | | [symbol] | | | | [symbol] | | [symbol] | | [symbol] |
| 06 | 37 | [symbol] % | [symbol] | | [symbol] | | [symbol] | [symbol] | [symbol] | [symbol] | [symbol] | | |
| 07 | 44 | [symbol] | | [symbol] | | | [symbol] | [symbol] | | [symbol] | [symbol] | | [symbol] |
| 08 | 4 [symbol] | [symbol] % | | [symbol] | | | | | | | | | |
| 09 | 30 | [symbol] % | | | | | | | [symbol] | | | | |
| 10 | 325 | [symbol] % | | | | | | | | | | | |
| 11 | 135 | [symbol] % | | | | | | | | | [symbol] | | |

| | PHLOROTANNIN DISTRIBUTION | | | | | | | | | | | | |
|---|---|---|---|---|---|---|---|---|---|---|---|---|---|
| | 10.1 | 10.2 | 10.5 | 10.6 | 10.9 | 11.1 | 11.4 | 11.6 | 11.9 | 12.3 | 12.4 | 12.6 | 12.7 |
| | min | min | min | min | min | min | min | min | min | min | min | min | min |
| CPC | DP | DP | DP | DP | DP | DP | DP | DP | DP | DP | DP | DP | DP |
| Fractions | 8 | 6 | 11 | 9 | 7 | 10 | 11 | 12 | 12 | 13 | 14 | 15 | 16 |
| 01 | [symbol] | [symbol] | [symbol] | | [symbol] | [symbol] | [symbol] | [symbol] | [symbol] | [symbol] | [symbol] | [symbol] | [symbol] |
| 02 | | [symbol] | [symbol] | [symbol] | [symbol] | [symbol] | [symbol] | [symbol] | [symbol] | [symbol] | [symbol] | [symbol] | [symbol] |
| 03 | [symbol] | | [symbol] | [symbol] | [symbol] | [symbol] | [symbol] | [symbol] | [symbol] | [symbol] | | [symbol] | [symbol] |
| 04 | [symbol] | | [symbol] | [symbol] | [symbol] | [symbol] | [symbol] | [symbol] | [symbol] | [symbol] | | [symbol] | [symbol] |
| 05 | [symbol] | | [symbol] | [symbol] | [symbol] | [symbol] | [symbol] | [symbol] | [symbol] | [symbol] | [symbol] | [symbol] | [symbol] |
| 06 | [symbol] | | [symbol] | | [symbol] | [symbol] | [symbol] | [symbol] | [symbol] | [symbol] | [symbol] | [symbol] | [symbol] |
| 07 | [symbol] | | [symbol] | | [symbol] | [symbol] | [symbol] | [symbol] | [symbol] | [symbol] | [symbol] | [symbol] | [symbol] |
| 08 | [symbol] | | [symbol] | | [symbol] | [symbol] | [symbol] | [symbol] | [symbol] | [symbol] | [symbol] | [symbol] | [symbol] |
| 09 | [symbol] | | [symbol] | [symbol] | [symbol] | [symbol] | [symbol] | [symbol] | [symbol] | [symbol] | [symbol] | [symbol] | [symbol] |
| 10 | | | [symbol] | | | | | | [symbol] | [symbol] | [symbol] | [symbol] | [symbol] |
| 11 | | | [symbol] | | | | | [symbol] | [symbol] | [symbol] | [symbol] | [symbol] | [symbol] |

DP = Polymerization degree of phlorotannins [symbol]
indicates data missing or illegible when filed

Other phlorotannins with higher polymerization degree were also present in the CPC fractions but not well detected by MS.

### EXAMPLE 3: IN TUBO EVALUATION OF ANTI-TYROSINASE ACTIVITY OF THE HIMANTHALIA ELONGATA EXTRACT

Unpurified and purified extract of Himanthalia elongata as obtained in example 1 have been tested for ability to inhibit the enzyme tyrosinase to identify interesting whitening activity for cosmetic applications.

With its natural substrate, the tyrosinase will lyse t-DOPA The presence of enzyme s inhibitors allows reducing the hydrolysis speed. The revelation of this activity corresponds to an absorbance measure (OD) of the dopaquinone released in the medium after hydrolysis of the substrate. When the enzyme is not inhibited, the concentration of dopaquinone released increases and a brown coloration as observed.

The results obtained are expressed in terms of IC50 (mg/ml). The half maximal inhibitory concentration (CS) is a measure of the effectiveness of a substance in inhibiting a specific biological or bio-chemical function. The lower the IC50, the stronger biological activity is at low concentrations of extract.

This assay is adapted to 96-wells microplate. 40 μL of sample are mixed with 60 μL of Phosphate buffer pH6.8. Then, 40 μl of tyrosinase are added. The reaction mixture is shaked and pre-incubated at 37° C. during 15 min. Then, 40 μL of L-DOPA solution are added. The microplate is incubated at 37° C. and read at a wavelength of 475 nm every 5 minutes during 15 minutes. Kojic acid is used as positive reference.

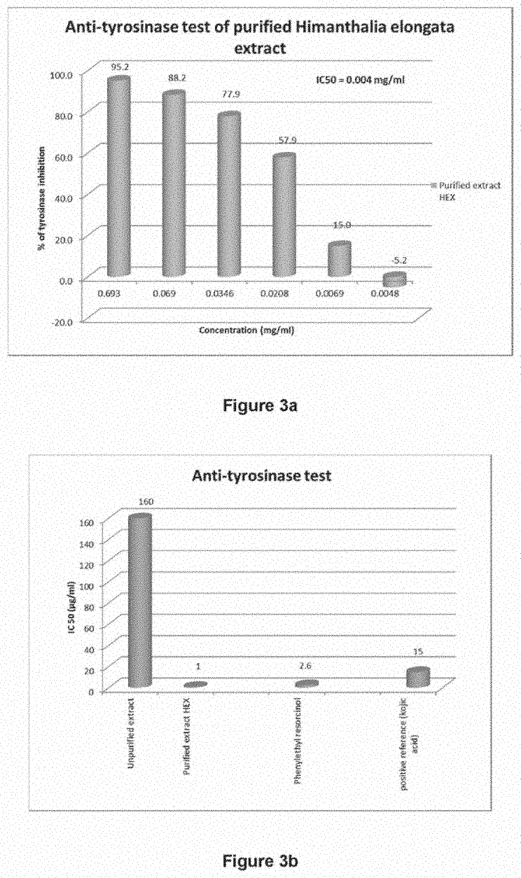

The purified extract has been tested at different concentrations. The results are displayed in FIG. 3a. The results obtained are expressed in % of inhibition of tyrosinase.

The results obtained on Himanthalia elongata extract (as obtained in Example 1a), on purified extract HEX (as obtained in example 1b with a higher content of polyphenols) vs Phenylethyl resorcinol are shown in FIG. 3b. The purified extract is 2.6 times more active than Phenylethyl resorcinol (benchmark) and 160 times more active than the unpurified extract. It was shown, that Himanthalia elongata extract can inhibit tyrosinase.

### EXAMPLE 4: IN TUBO EVALUATION OF ANTI-HYALURONIDASE ACTIVITY OF THE HIMANTHALIA ELONGATA EXTRACT

The evaluation of the anti-hyaluronidase activity by turbidimetric method is based on the formation of a precipitate obtained by putting the hyaluronic acid polymer in contact with a surfactant, the CTAB (cetyltrimethylammonium bromide).

During the enzymatic reaction, hyaluronic acid (formed from 20 to 12,500 units) is degraded by hyaluronidase to give polymers of reduced size. Thus, the obtained disaccharides and polymers of sizes smaller than 8 kDA do not precipitate in the presence of CTAB, while the polymers of undegraded hyaluronic acid and of sizes greater than 8 kDA precipitate in contact with CTAB.

The assay has been performed in 96 wells microplate. 20 μl of sample are first mixed with 10 μl of Mac Ilvaine's buffer pH 4.6. Then 20 μl of hyaluronidase and 20 μl of hyaluronic acid are added. The mixture is shaked and incubated for 40 min at 37° C. Finally 180 μl of CTAB are added in all wells to precipitate non-hydrolyzed hyaluronic acid polymer. The mixture is incubated for 20 min at room temperature, and the absorbance at 600 nm is measured by spectrophotometry. DSCG (disodium cromoglycate) is used as positive control.

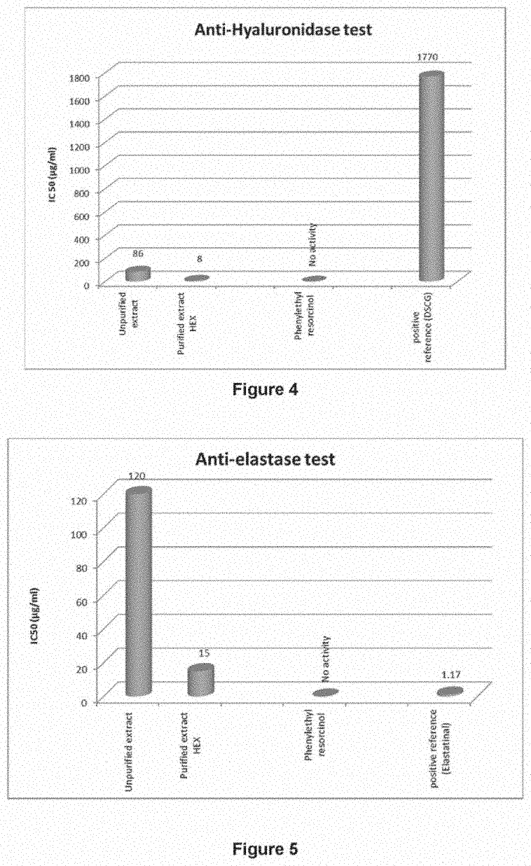

The results obtained on Himanthalia elongata extracts unpurified and purified in phlorotannins (as obtained in example 1a and 1b, respectively) vs Phenylethyl resorcinol are shown in FIG. 4. It was shown, that the purified extract of Himanthalia elongata has the best anti-hyaluronidase activity as well as the global extract of Himanthalia elongata have a significant anti-hyaluronidase activity. It was shown, that Himanthalia elongata extract can inhibit hyaluronidase.

### EXAMPLE 5: IN TUBO EVALUATION OF ANTI-ELASTASE ACTIVITY OF THE HIMANTHALIA ELONGATA EXTRACT

In contact with its natural substrate, the elastase will lyse the N-succinyl-(Ala)3-p-nitroanilide. The presence of inhibitors of this enzyme makes it possible to reduce the rate of hydrolysis.

The revelation of this activity corresponds to an absorbance measurement (OD) of the p-nitroaniline released in the medium after hydrolysis of the substrate. When the enzyme is not inhibited, the concentration of p-nitroaniline released increases and a yellow color appears.

The anti-elastase assay employed was based on spectrophotometric method and is performed in 96 wells microplate. The porcine pancreatic elastase is used as enzyme and N-succinyl-(Ala)3-p-nitroanilide as the substrate, monitoring the release of p-nitroanilide. The reaction mixture contains 25 μL of sample, 25 μL of 0.2M Tris-HCl buffer (pH 8), 25 μL of enzyme elastase. The mixture is pre-incubated at 37° C. during 15 min. Then, 100 μL of N-succinyl-(Ala)3-p-nitroanilide as the substrate are added. The absorbance is monitored at 405 nm after 30 min of incubation at 37° C. Elastatinal is used as positive control.

The results obtained on different Himanthalia elongata extracts unpurified and purified in phlorotannins (as obtained in example 1a and 1b, respectively) vs Phenylethyl resorcinol are shown in FIG. 5. It was shown, that the purified extract of Himanthalia elongata has the best anti-elastase activity and the unpurified extract of Himanthalia elongata have a significant anti-elastase activity. It was shown, that Himanthalia elongata extract can inhibit elastase.

### EXAMPLE 6: IN TUBO EVALUATION OF ANTI-COLLAGENASE ACTIVITY OF THE HIMANTHALIA ELONGATA EXTRACT

The estimation of the anti-aging activity is carried out by determining the ability of the extract to delay the enzymatic reaction of the collagenase of the bacterium Clostridium histolyticum (ChC) with a fluorescent peptide substrate (MMP-2), by spectrofluorimetry at 320 nm (excitation) and 405 nm (emission). PADS (phosphoramidon disodium sulfate) is used as positive reference.

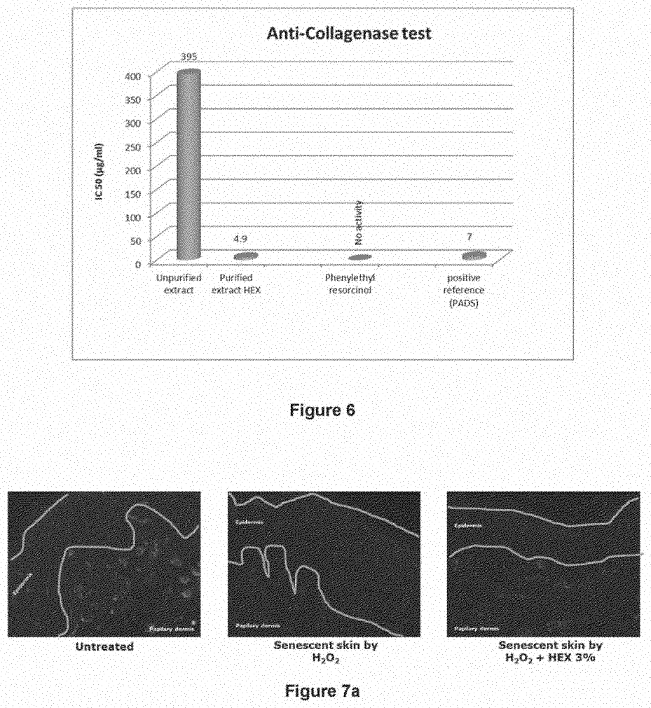

The results obtained for Himanthalia elongata extracts (as obtained in example 1a and 1b, respectively) vs Phenylethyl resorcinol are shown in FIG. 6. It was shown, that the purified extract of Himanthalia elongata as well as the unpurified extract of Himanthalia elongata have a significant anti-collagenase activity. It was shown, that Himanthalia elongata extract can inhibit collagenase.

### EXAMPLE 7; EX VIVO TESTING ON AGEING SPOTS—SENILE LENTIGO

i) Skin Explants Treatment

Dermal fibroblasts senescence was induced to human fresh skin explants (from woman donor, aged 35 years) by a chemical systemic treatment with H₂O₂ at 200 μM for 2 hours. To evaluate the impact of a topical application with Himanthalia elongata extract, the explants have been treated as follows:

After senescence induction, skin medium was renewed with fresh medium, and a) skin explants were topically treated with 3% (v/v) Himanthalia elongata extract (purified extract), or b) the skin explants were not treated for untreated senescent condition Other skin explants for the untreated basal control were not treated, and their medium was just renewed daily.

These conditions were daily repeated for 5 days. After sampling, skin explants were fixed in formalin before being dehydrated and embedded in paraffin.

ii) Hyperpigmentation (SDF1)

Method: SDF1 Immunostaining

Explants embedded in paraffin were cut 4 μm thick before being dewaxed. Antigenic retrieval was performed in EDTA (Ethylenediaminetetraacetic acid) pH8 buffer before saturating the skin samples' aspecific sites with bovine serum albumin. Samples were then incubated with anti-SDF1 antibody overnight at 4° C., followed by rinses and another incubation with secondary antibody coupled to Alexa Fluor 488 for 45 min at room temperature in the darkness. Samples were rinsed and assembled with coverslips and mounting medium containing DAPI (4′,6-diamidino-2-phenylindole). Positive cells for SDF1 expression were scored.

Results: Control of Hyperpigmentation Through SDF1 Expression

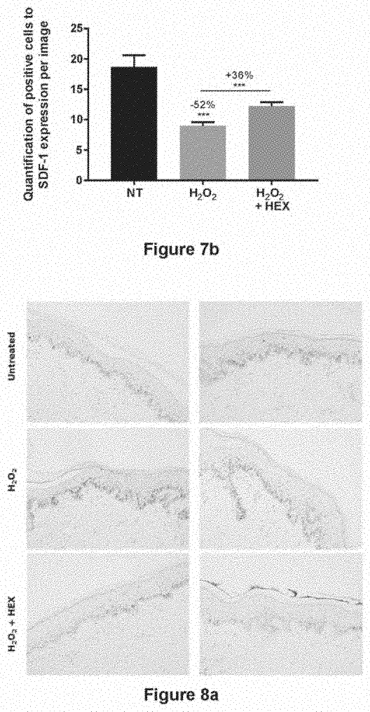

By comparison with untreated skin (FIG. 7a, left) it was confirmed that the senescence induction significantly reduced the number of fibroblasts expressing SDF1 (FIG. 7a, middle), which means a loss of pigmentation control. After the senescent explants treatment with HEX at 3% for 5 days a significant increase of SDF1 expression was observed (FIG. 7a, right), leading to a potential activation of the pigmentation control.

Also scoring of a number of fibroblasts expressing SDF1 near the dermo epidermal junction for each condition (FIG. 7b) confirmed said result.

It was shown, that Himanthalia elongata extract can increase SDF1 expression, thereby activating pigmentation control, in particular in relation to Senile lentigo.

iii) Hyperpigmentation (Fontana's Masson Staining)

Method: Melanin Quantification Using Fontana's Masson Staining and Imaging Analysis

The formalin-fixed and paraffin-embedded explants were cut 4 μm thick before being dewaxed and then stained by Fontana Masson silver method for melanin. Images were collected with Axio Observer Inverted fluorescence microscope (Zeiss) in bright field mode.

Melanin was quantified using two open source optical imaging software programs. Photomicrographs (jpeg format) of Fontana-Masson tissue sections were opened in GIMP-GNU Image Manipulation Program. The brown-black colour signals corresponding to melanin grains on stained sections were selected, copied and pasted into a new image and saved as a jpeg file; this jpeg file consists solely of black/brown (melanin grains) on a white background. This image was subsequently opened using the ImageJ program. A histogram of the image was created which separates the total number of pixels in the image into 255 colour categories spanning the visible spectrum. The peak corresponding to the brown-black colour (melanin) was determined by cutting and summing the appropriate counts from each channel of the melanin peak. Alternatively, the numbers corresponding to the pigment peak could be pasted into an Excel spreadsheet and summed. The pigmentation index was obtained by taking the absolute number of black pixels in a representative 20× field as determined by Image J and multiplying it by 10⁻³.

Results: Control of Hyperpigmentation Using Fontana's Masson Staining and Imaging Analysis

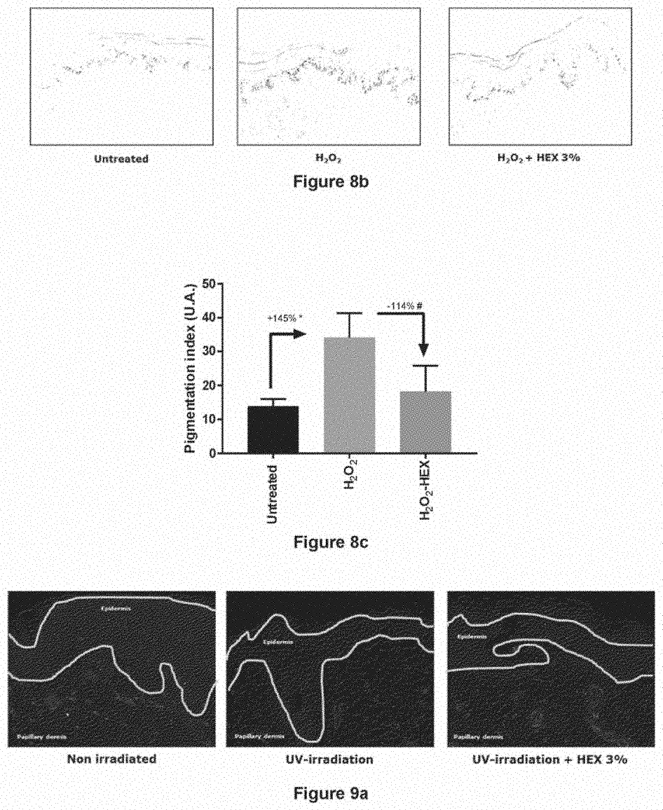

On the same skin explants. Fontana's Masson staining was performed to visualize the melanin content before and after application (FIG. 8a).

It was evidenced that H₂O₂ has induced an important increase of melanin content in comparison with untreated condition, through the inhibition of SDF1 expression. This result demonstrated that the model mimic the hyper-pigmentation found in senile lentigo.

Topical application of HEX at 3% for 5 days sharply reduce the melanin content as the same level as what we observed in untreated condition, probably thanks to the reactivation of SDF1 expression by senescent fibroblasts.

The calculation of the pigmentation index using imaging analysis (FIG. 8b) demonstrated that there is a significant hyper pigmentation with the senescent induction as observed by the results in presence of H₂O₂ with +145%. A topical application of HEX at 3% for 5 days leads to a significant reduction of melanin biogenesis as observed by the reduction of melanin content (FIG. 8c).

### EXAMPLE 8; EX VIVO TESTING ON AGEING SPOTS—SOLAR LENTIGO

i) Skin Explants Treatment

Human fresh skin explants (from woman donor, aged 35 years) were daily irradiated with UV-A (9 J/cm²) and UV-B (0.33 J/cm², ratio UV-A/UV-B=27) for 5 days for a total of 5 single irradiations. After each single irradiation, three explants were topically treated with 3% (v/v) Himanthalia elongata extract (purified extract), and other explants were not treated for untreated UV exposure conditions. The explants of the control group did not receive any irradiation or treatment. After sampling, explants were cut in two parts, one part preserved in OCT and used to perform carbonyl score imaging and the other part fixed in formalin before being dehydrated and embedded in paraffin for Fontana Masson staining and complementary immunochemistry. Supernatant was collected the last day 8 hours post-irradiation in order to measure IL-8 production.

ii) Hyperpigmentation (SDF1)

For method, see example 7ii)

Results: Control of Hyperpigmentation Through SDF1 Expression

Daily UV exposure is described in the literature to lead to skin premature ageing through the induction of skin's cells senescence. By performing a SDF1 immunostaining the impact of UV-induced senescence on the SDF1 expression was demonstrated. It was also shown, how Himanthalia elongata extract can change this phenomenon.

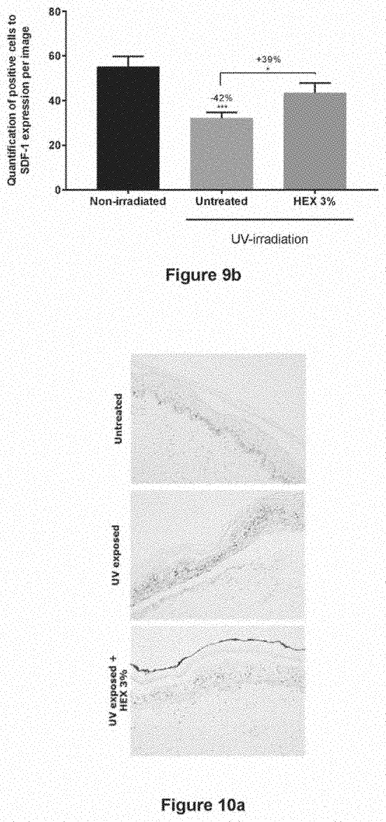

By comparison with untreated skin (FIG. 9a, left) it was confirmed that the UV exposure significantly reduced the number of fibroblasts expressing SDF1 (FIG. 9a, middle), which means a loss of pigmentation control. After the UV-irradiated explants treatment with HEX at 3% for 5 days a significant increase of SDF1 expression was observed (FIG. 9a, right), leading to a potential activation of the pigmentation control.

Also scoring of a number of fibroblasts expressing SDF1 near the dermo epidermal junction for each condition (FIG. 9b) confirmed said result. It was shown, that Himanthalia elongata extract can increase SDF1 expression, thereby activating pigmentation control, in particular in relation to Solar lentigo.

iii) Hyperpigmentation (Fontana's Masson Staining)

For method, see example 7iii)

Results: Control of Hyperpigmentation Using Fontana's Masson Staining and Imaging Analysis

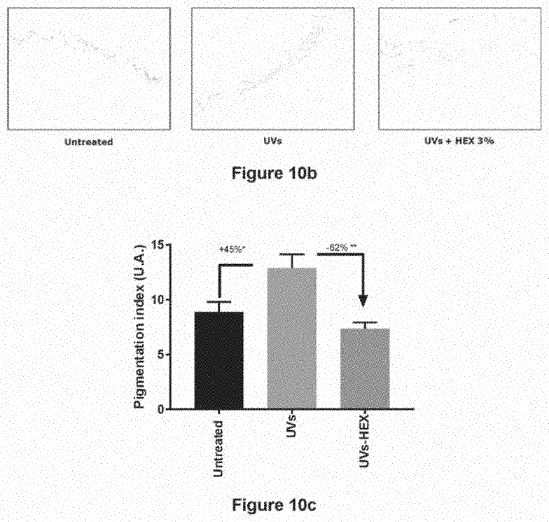

It was shown that the UV exposure induced an increase of melanin content in skin explant as observed using Fontana's Masson staining in comparison with untreated condition (see FIG. 10a top and middle). A topical application of HEX at 3% for 5 days reduced the UV-induced hyperpigmentation (FIG. 10a bottom).

The calculation of the pigmentation index using imaging analysis (FIGS. 10b, 10c) demonstrated that there is a significant hyperpigmentation induced by the UV exposure which is significantly reduced by a topical application of Hex at 3% after 5 days.

It was shown, that Himanthalia elongata extract can reduce hyperpigmentation induced by the UV exposure.

iv) Reduction of Oxidized Proteins

Method: Quantification of Oxidized Proteins by Carbonyl Score Measurement

Explant sections of 4 μm thickness were obtained using a cryostat (Leica) and fixed with a solution containing 95% ethanol and 5% acetic acid. Carbonylated proteins were labelled using a specific fluorescent probe (Ex=480 nm/Em=530 nm) that reacts with carbonyl moieties and DAPI (4′,6-diamidino-2-phenylindole) for nuclear labelling.

Fluorescent images were collected with an epifluorescent microscope (EVOS M5000 Imaging System; Thermo Fisher Scientific) and analyzed with ImageJ software (Schneider, 2012). Image comparisons of different conditions were achieved using identical conditions of acquisition for magnification (10× or 60× objectives).

Results: Reduction of Oxidized Proteins after UV Exposure

It was evidenced that the UV exposure induced an increase of oxidized proteins as observed by the intensity of red fluorescent detection. This effect is correlated to a decrease of proteasome activity which is responsible for detection and elimination of oxidized proteins.

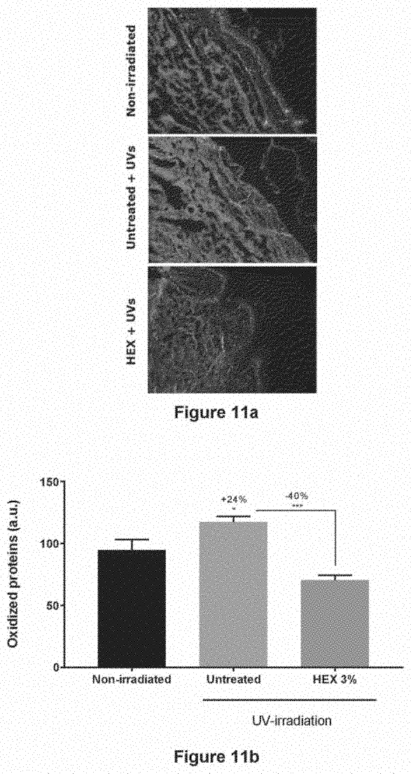

After 5 days of topical application of HEX at 3%, a significant reduction of oxidized proteins was observed. Therefore, it was demonstrated that HEX was able to reactivate proteasome activity which is drastically affected in solar lentigo (FIG. 11a).

The quantification of the signal confirmed the observation and showed a significant increase of oxidized proteins by +24% after UV exposure which is significantly reduced by topical application of HEX at 3% with −40% (FIG. 11b).

It was shown, that Himanthalia elongata extract can reduce the presence of oxidized proteins after UV exposure.

v) Reduction of Lipofuscin Bodies Accumulation after UV Exposure

Method: Sudan Black B Staining

Cryosections of 8 μm thickness were obtained using a cryostat and fixed with 10% formalin for 10 minutes. After fixation, coloration was processed in successive bathes of absolute propylene glycol for 12 minutes, 0.7% Sudan Black B solution prepared in absolute propylene glycol for 15 minutes at 60° C., followed by a rinsing in propylene glycol 85° and three distilled water bathes. Coverslips were added on cryosections with an aqueous mounting medium and images were collected with Axio Observer Inverted fluorescence microscope (Zeiss) in bright field mode.

Quantification of Lipofuscin Accumulation:

Lipofuscin was quantified using two open source optical imaging software programs. Photomicrographs (Tif format) of Sudan Black B tissue sections were opened in Image J software and color channels were splited. Green channel which is the more representative of the Sudan Black B staining was selected and saved before being opened in GIMP—GNU Image Manipulation Program. The dark colour signals corresponding to lipofuscin on stained sections were selected, copied and pasted into a new image and saved as a jpeg file; this jpeg file consists solely of dark lipofuscin on a white background. This image was subsequently opened using the ImageJ program and inverted to get the background in black and the lipofuscin staining in white which corresponds to the signal to be measured. The staining intensity was measured in the epidermis area, giving a value corresponding to an amount of lipofuscin.

Results: Reduction of Lipofuscin Bodies Accumulation after UV Exposure

It was evaluated whether the accumulation of oxidized proteins could lead to the formation of lipofuscin bodies in the epidermis. Sudan Black B staining was used which is specific to detect the lipids in order to visualize the lipofuscin accumulation after UV exposure (Evangelou et al. 2017).

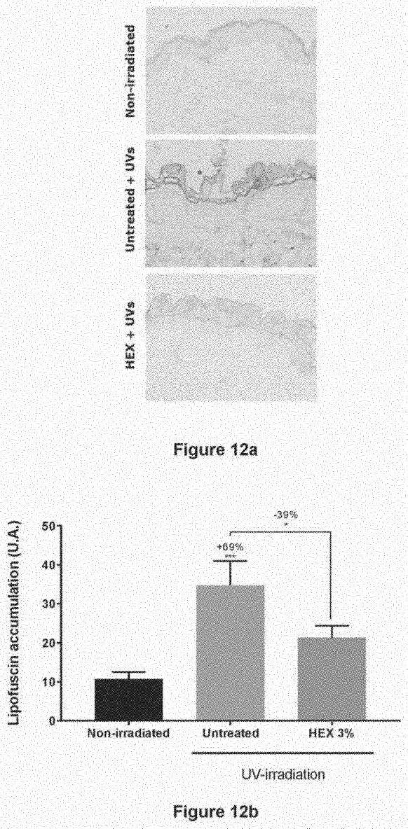

The Sudan Black B staining evidenced a significant increase of lipofuscin bodies accumulation by +69% after UV exposure. This is clearly visible by the increasing of blue staining after UV exposure in comparison with untreated condition (FIG. 12a top and middle, FIG. 12b)).

A topical application of HEX at 3% significantly reduced UV-induced lipofuscin bodies accumulation by −39% after 5 days that is also observed by reduction of blue staining in pictures (FIG. 12a bottom, 12b).

This result shows that HEX has a protective effect against lipid oxidation allowing decreasing the lipofuscin bodies accumulation in the epidermis, which is a marker of skin ageing. It was shown, that Himanthalia elongata extract can reduce accumulation of lipofuscin bodies after UV exposure.

vi) Pro-Inflammatory Cytokine (IL-8)

Method: Quantification of Pro-Inflammatory Cytokine (IL-8)

Supernatant of skin explant were diluted 1.5 for those from non-irradiated explants and diluted 1:20 for those from irradiated explants IL-8 release from explants was quantified by an ELISA dosage using Human IL-8 Quantikine ELISA kit (Biotechne). ELISA has been done following supplier instructions. Optical density resulting from this colorimetric assay was read with Tecan microplate reader (Spark).

Results: Reduction of Inflammation after UV Exposure

An increase of inflammatory response has been observed especially through the quantification of pro-inflammatory cytokine IL-8 which has evidenced a strong increase with UV exposure with +121%.

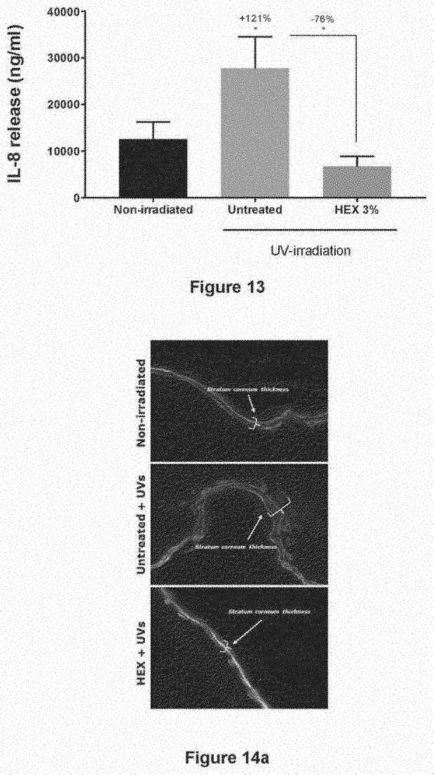

A topical application of HEX at 3% significantly and drastically reduced the UV-induced over-production of IL-8 demonstrating that HEX was able to control the inflammation related to solar lentigo (FIG. 13).

It was shown, that Himanthalia elongata extract can reduce inflammation after UV exposure.

vii) Reduction of Stratum Comeum Thickness after UV Exposure

Method: Filaggrin Immunostaining

Explants embedded in paraffin were cut 4 μm thick before being dewaxed. Antigenic retrieval was performed in EDTA pH8 buffer before saturating the skin samples' aspecific sites with bovine serum albumin, Samples were then incubated with anti-filaggrin antibody for 2 hours at room temperature, followed by rinses and another incubation with secondary antibody coupled to Alexa Fluor 488 for 1 hour at room temperature in the darkness. Samples were rinsed and assembled with coverslips and mounting medium containing DAPI (4′,6-diamidino-2-phenylindole). Stratum comeum thickness was measured by ImageJ software following the specific filaggrin staining allowing visualizing stratum comeum.

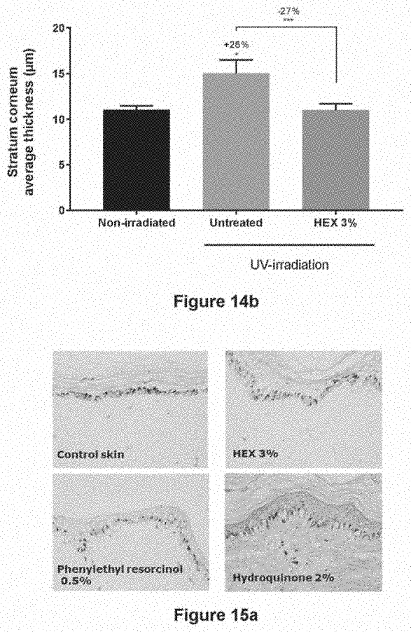

Results: It was shown that UV exposure had an impact on skin barrier integrity due an increase of stratum comeum thickness translating a loss of cohesion between comeocytes. By observing the filaggrin immunostaining it was shown that a topical application of HEX at 3% has significantly decreased the stratum comeum thickness as followed by filaggrin expression (green fluorescence, FIG. 14a).

Indeed, a thickening from 11 μm to 15 μm after UV exposure with +26% was shown. It was observed that a topical application of HEX at 3% has restored the stratum comeum thickness close to the non-irradiated condition showing 11 μm of thickness with −27% (FIG. 14b). It was shown, that Himanthalia elongata extract can reduce Stratum comeum thickness after UV exposure.

### EXAMPLE 9: EX VIVO TESTING OF WHITENING EFFICACY

i) Skin Explants Treatment

Skin fragments from 3 Asian, 3 African and 3 Caucasian subjects were cut into 1 cm² pieces and washed three times using antibiotics. Subcutaneous fat and lower dermis were mechanically removed under a stereomicroscope using a surgical scalpel.

Skin biopsies were placed with the epithelium uppermost at an air/liquid interface on culture inserts (filter pore size 12 μm; Costar, VWR International, Fontenay-sous-Bois, France). These inserts were set on twelve wells plate for 14 days at 37° C. in a humidified incubator with 5% CO₂.

Medium was added to the wells so that the surface of the medium was levelled with the filter. Organ cultures were conserved with Dulbecco's minimal essential medium (Invitrogen Corporation, Paisley, UK) containing antibiotic, L-glutamine, bovine pituitary extract, growth factors and fetal calf serum (DAP, Neuf-Brisach, France).

Himanthalia elongata extract 3% (unpurified extract), Phenylethyl resorcinol 0.5% and hydroquinone 2% (SIGMA H9003) as positive control were topically applied daily during 14 days on the epidermis of the skin fragments, in comparison with untreated skin.

4 conditions (realized in triplicate) have been compared:
untreated skin
skin treated by Himanthalia elongata extract 3% (v/v)
skin treated by Phenylethyl resorcinol 0.5% (w/v)
skin treated by hydroquinone 2% (w/v).

ii) Method: Histological Analysis by Fontana's Masson Staining and Whitening Scoring by Expert

For testing the depigmentation product, the melanin pigment present in the melanocytes and in the contiguous keratinocytes of the basal cell layer of the epidermis from treated skin in comparison with untreated skin is evaluated.

Samples were formalin-fixed at day 14 and paraffin-embedded Sections (4 μm) were then stained by Fontana Masson silver method for melanin (see example 4iii).

A quantitative numeration of cells containing melanin pigments was made under an optical microscope at ×40 magnification on about 300 basal cells of the epidermis, Two types of cells were counted:
score 1: unpigmented cells or cells presenting rare melanin pigments isolated in the cytoplasm
score 3: cells presenting important melanin pigment in all over the cytoplasm

Epidermal melanin content was calculated as the percentage of the epidermal cells in each scoring.

Results:

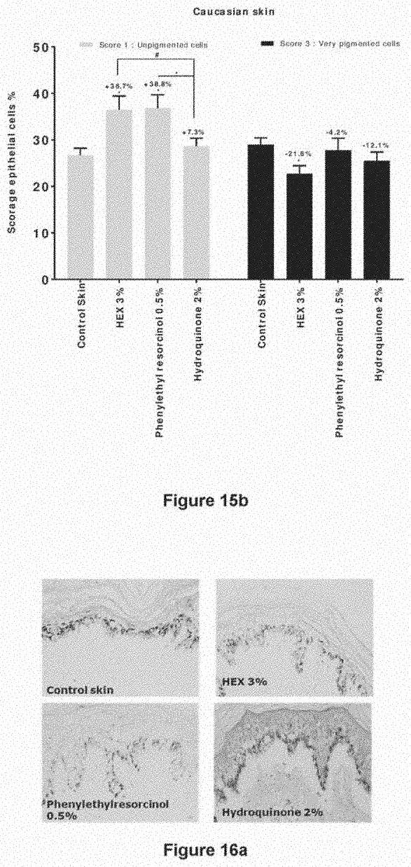

a) Whitening on Caucasian Skin Explants (FIG. 15a,b)

A topical application of unpurified Himanthalia elongata extract at 3% over 14 days significantly reduced the percentage of very pigmented cells (−21.6%) and significantly increased the unpigmented cells (+36.7%) on Caucasian skin explants, demonstrating the whitening efficacy of Himanthalia elongata extract at 3% on Caucasian skin.

In comparison, Phenylethyl Resorcinol only significantly increase the percentage of unpigmented cells (+38.8%), but there was no impact on very pigmented cells (−4.2%). It can be concluded that Himanthalia elongata extract 3% is more efficient than Phenylethyl Resorcinol on Caucasian skin explants.

Himanthalia elongata extract at 3% is also significantly better than Hydroquinone at 2% which didn't show any significant impact on unpigmented or very pigmented cells on Caucasian skin.

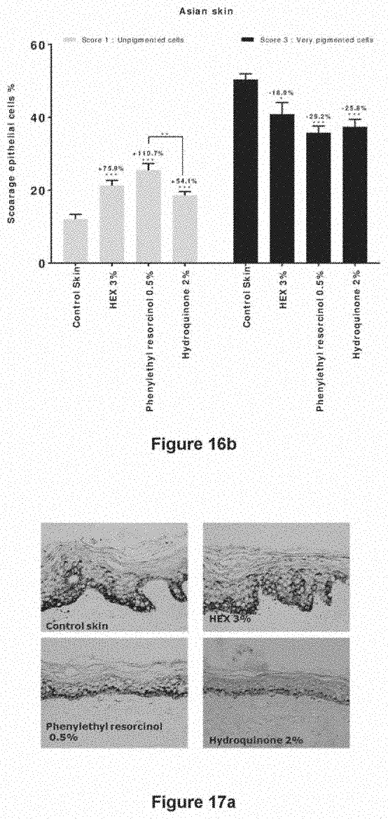

b) Whitening on Asian Skin Explants (FIG. 16a,b)

A topical application of unpurified Himanthalia elongata extract at 3% over 14 days significantly reduced the percentage of very pigmented cells (−18.9%) and significantly increased the unpigmented cells (+75.9%) on Asian skin explants, demonstrating the whitening efficacy of Himanthalia elongata extract at 3% on Asian skin.

The topical application of Phenylethyl Resorcinol significantly increased the percentage of unpigmented cells (+110.7%) and significantly decreased the percentage of pigmented cells (−29.2%). Therefore, Himanthalia elongata extract 3% is as efficient as Phenylethyl Resorcinol on Asian skin explants.

Himanthalia elongata extract at 3% is also as efficient on whitening activity as Hydroquinone at 2% which caused a significant increase of unpigmented cells (+54.1%) and a significant decrease if very pigmented cells (−25.8%) on Asian skin.

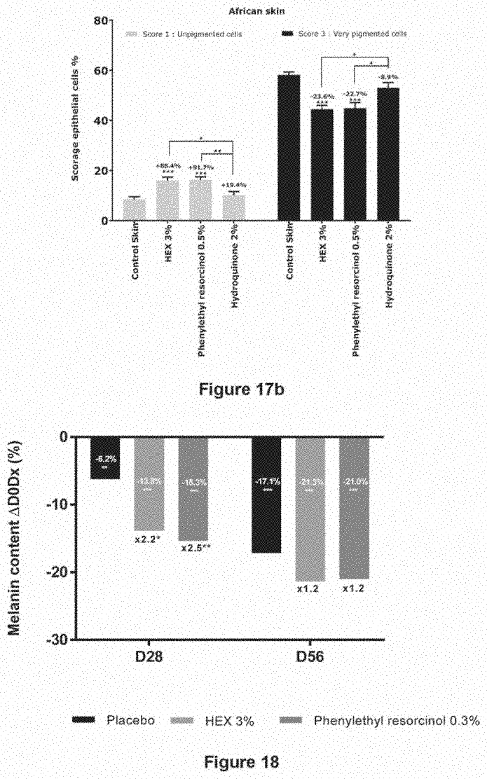

c) Whitening on African Skin Explants (FIG. 17a,b)

A topical application of unpurified Himanthalia elongata extract at 3% over 14 days significantly reduced the percentage of very pigmented cells (−23.6%) and significantly increased the unpigmented cells (+88.4%) on African skin explants, demonstrating the whitening efficacy of Himanthalia elongata extract at 3% on African skin.

The treatment with Phenylethyl Resorcinol also significantly increase the percentage of unpigmented cells (+91.7%) and significantly decreased the percentage of pigmented cells (−22.7%), showing that Himanthalia elongata extract 3% is as efficient as Phenylethyl Resorcinol on African skin explants.

However, Himanthalia elongata extract at 3% is more efficient on whitening activity than Hydroquinone at 2% which didn't show any significant results on unpigmented cells and on very pigmented cells after topical application on African skin.

It was shown, that Himanthalia elongata extract has a whitening effect on different types of skin.

### EXAMPLE 10: CLINICAL TESTING—REDUCTION OF PIGMENTED SPOTS ON HANDS OF CAUCASIAN VOLUNTEERS

i) INCI Formula Used

| | | Phenylethyl, | |
|---|---|---|---|
| | Placebo | Resorcinol | HEX |
| | formula | formula | formula |
| INCI Name | (%) | (%) | (%) |
| AQUA/WATER | 89.70 | 89.40 | 86.70 |
| CETYL ALCOHOL, GLYCERYL | 5.0 | 5.0 | 5.0 |
| STEARATE, PEG-75 STEARATE, | | | |
| CETETH-20, STEARETH-20 | | | |
| ISODECYL NEOPENTANOATE | 4.5 | 4.5 | 4.5 |
| PHENYLETHYL RESORCINOL | — | 0.3 | — |
| HIMANTHALIA ELONGATA | — | — | 3.0 |
| EXTRACT (purified extract) | | | |
| PHENOXYETHANOL, METHYL | 0.4 | 0.4 | 0.4 |
| PARABEN, PROPYL PARABEN, | | | |
| ETHYL PARABEN | | | |
| DIMETHICONE | 0.3 | 0.3 | 0.3 |
| FRAGRANCE, BENZYL | 0.1 | 0.1 | 0.1 |
| SALICYLATE, LINALOOL, D- | | | |
| LIMONENE | | | |

ii) Panel Description

The study was a double-blind, randomized and placebo controlled study. The assessment was based on an intra-subject comparison between the results obtained with cream HEX at 3% (active product), cream Phenylethyl resorcinol at 0.3% and the cream placebo. The measurements were taken in controlled-atmosphere at a temperature of 21±1° C. and a humidity of 50*10%. This study was performed on 39 volunteers (healthy Caucasian woman aged from 50 to 75 years, mean age: 61.7±5.6 years) having pigmented ageing spots on their hands.

iii) Treatment

The study was carried out on 2 groups of around 20 volunteers each (39 volunteer in total aged from 50 to 70 years). A cream formula was applied twice daily on hands. Evaluation was carried out before treatment and after 28 and 56 days. Group 1 has applied placebo on one hand and HEX at 3% on the other hand, and group 2 has tested HEX at 3% on one hand versus Phenylethyl Resorcinol at 0.3% on the other hand.

iv) Method: Mexameter® Analysis

The measurement is based on the absorption principle. The MEXAMETER MX 18 probe emits radiation of three defined wavelengths. A receiver measures the reflection of the light reflected by the skin. The position of the transmitter and receiver ensures that only diffuse light is measured. With a defined amount of emitted light the amount of light absorbed by the skin can be calculated. Melanin is measured by two wavelengths. The wavelengths were chosen according to the different absorption peaks of the melanin pigments. For the measurement of erythema (redness of skin), two different wavelengths are used to measure the absorption capacity of the epidermis. One of them corresponds to the absorption spectrum of haemoglobin. The other wavelength was chosen in order to have other pigments influencing the colour of the skin.

v) Results:

a) Reduction of Melanin Content (FIG. 18)

HEX at 3% was able to significantly reduce the melanin content of the skin after 28 days and 56 days with −13.8% and −21.3% respectively relative to D0.

The placebo cream caused a slight whitening effect during the study, but HEX at 3% significantly more reduced the melanin content than placebo showing a 2.2 times more efficient whitening activity after 28 days of HEX's application.

Same melanin reduction efficacy as with HEX at 3% was obtained with Phenylethyl Resorcinol 0.3%, as no significant difference between the both products was observed.

It was shown, that Himanthalia elongata extract can reduce melanin content of the skin, thereby reducing pigmented spots on skin, in particular on hands of Caucasian volunteers.

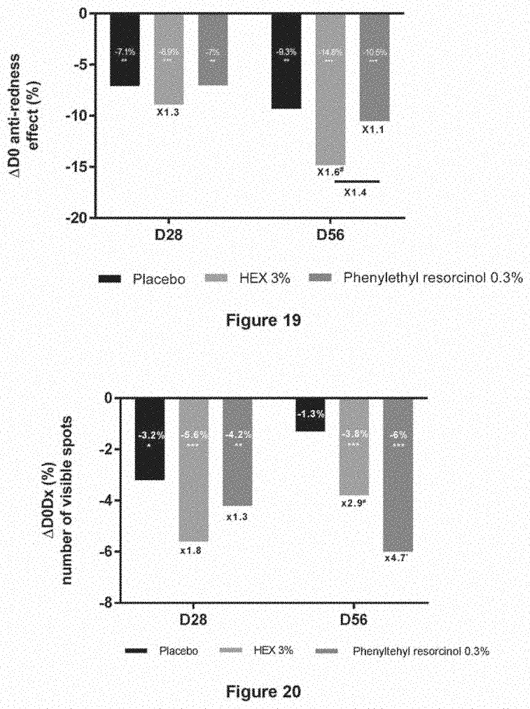

b) Reduction of Skin Redness (FIG. 19)

HEX at 3% was able to significantly reduce the skin redness of the skin after 28 days and 56 days with −8.9% and −14.8% respectively relative to D0.

Placebo caused a slight reduction of redness during the study, but HEX at 3% was significantly more efficient on the reduction of skin redness than placebo showing a 1.6 times more efficient redness reduction after 56 days with HEX's application in comparison with placebo.

Phenylethyl Resorcinol evidenced the same efficacy as what we obtained with the placebo. This result demonstrated that HEX is more efficient than Phenylethyl resorcinol on the reduction of skin redness on pigmented ageing spots on the hands.

It was shown, that Himanthalia elongata extract can reduce skin redness on pigmented ageing spots, in particular on hands of Caucasian volunteers.

vi) Illustrative Pictures Using DermaScop®

Illustrative pictures were taken by Dermascop® on specific pigmented ageing spots on hand before and after products application. These pictures were used only for qualitative appreciation of the effect. A clear positive effect of HEX at 3% on the reduction of ageing pots similarly as the phenylethyl resorcinol efficacy at 0.3% was visualized. The placebo didn't show any positive impact on it.

### EXAMPLE 11: CLINICAL TESTING—REDUCTION OF PIGMENTED SPOTS ON FACE OF ASIAN VOLUNTEERS

i) INCI Formula Used

The formula described in example 10i) has been used.

ii) Panel Description

The study was a double-blind, randomized and placebo controlled study. The assessment was based on an inter-subject comparison between the results obtained with cream HEX at 3% (active product), cream Phenylethyl resorcinol at 0.3% and the cream placebo. The measurements were taken in controlled-atmosphere at a temperature of 21±1° C. and a humidity of 50±10%. This study was performed on 72 volunteers (healthy Asian woman aged from 45 to 85 years), having pigmented ageing spots on their faces.

iii) Treatment

The study was carried out on 2 groups of 36 volunteers each (72 volunteer in total). A cream formula was applied twice daily on face. Evaluation was carried out before treatment and after 28 and 56 days. Group 1 has applied placebo on one side of the face and HEX at 3% on the other side of the face Group 2 has tested HEX at 3% on one side of the face versus Phenylethyl Resorcinol at 0.3% on the other side of the face.

iv) Method: Ageing Spot Analysis by VISIA CR2.0

The device VISIA_{R} from CANFIELD® imaging systems has been used. It allows taking pictures with different types of illumination and a very rapid capture of images. The control of the repositioning takes place directly on data-processing screen using an overlay visualization of the images at each time of acquisition.

A series of photos taken under multi-spectral imaging and analysis (white light, UV or polarized light—parallel or crossed) allows capturing visual information affecting complexion health and appearance:

Under standard light, visible spots can be evaluated, in particular by visualisation of brown or red skin lesions including freckles, acne scars, hyper-pigmentation and vascular lesions. With cross polarized light in combination with Canfield's RBX® Technology brown spots can be evaluated, in particular by analysis of subsurface melanin conditions. Canfield's RBX® Technology separates the unique colour signatures of Red and Brown skin components for unequaled visualization of conditions that result in colour concentration, such as spider veins, hyperpigmentation, inflammation and other conditions. The results are given in number of lesions, which is a count of lesions detected by the device, regardless of their size or intensity.

v) Results:

a) Reduction of Visible Spots on Face (FIG. 20)

Using VISIA pictures, the number of visible spots has been analysed.

HEX at 3% significantly reduced the number of visible spots after 28 and 56 days of application with −5.6% and −3.8% respectively relative to D0.

Placebo cream slightly reduced the number of visible spots but it is clearly less efficient than cream containing HEX at 3%. Moreover, HEX at 3% cause significant reduction of visible spots in comparison with placebo with an efficacy 2.9 times higher after 56 days of HEX cream application.

The cream containing Phenylethyl Resorcinol at 0.3% has an efficacy very close to those obtained with HEX at 3% without significant difference between the both products indicating that HEX is as efficient as Phenylethyl resorcinol on the visible spots reduction on face area.

It was shown, that Himanthalia elongata extract can reduce visible spots on face, in particular of Asian volunteers.

b) Reduction of Brown Spots on Face (FIG. 21)

Brown spots which are related to the pigmented ageing spots on face have been evaluated by means of VISIA analysis.

The cream containing HEX at 3% significantly decreased the brown spot after 28 and 56 days of application with −3.4% and −2.7% respectively relative to D0. In addition, HEX at 3% is significantly more efficient than placebo with a significant reduction of brown spots by 4.9 times after 28 days of HEX cream.

In comparison with Phenylethyl resorcinol at 0.3%, it was shown that an activity slightly better than HEX on the reduction of brown spots on face without significant difference between both products, indicating that HEX at 3% is as efficient as Phenylethyl resorcinol on the reduction of brown spots on face. It was shown, that Himanthalia elongata extract can reduce brown spots on face, in particular of Asian volunteers.

vi) Method: Spectrocolorimeter Measurement

Skin colorimetric measurement was done with a MINOLTA CM700-d Spectrophotometer® equipped with a 8 mm diameter head. The Spectrophotometer® converts colours perceived by human to a digital code composed of three parameters:

L*: for clarity (from dark to light),

a*: for the green-to-red spectrum,

b*: for the blue-to-yellow spectrum.

a* and b* are chrominance parameters and L* is a luminance parameter.

It is possible to describe slightest differences between two cutaneous zones that appear to be the same colour. After a calibration phase, measurements are done directly on the skin using a pulsed Xenon light source and a dual beam system designed to measure the light transmitted and to correct any slight deviation.

The parameters b* (cutaneous melanin yellow colour) is studied during studies on pigmentary blotches. b* parameter is exploited through the calculation of the Individual Typological Angle (ITA°), which defines the skin pigmentation degree of a subject with taking into account the skin clarity (L*) and the melanin parameter (b*) according to the following formula:

ITA°=[Arc tan((L*·50)/b*)]×180/π

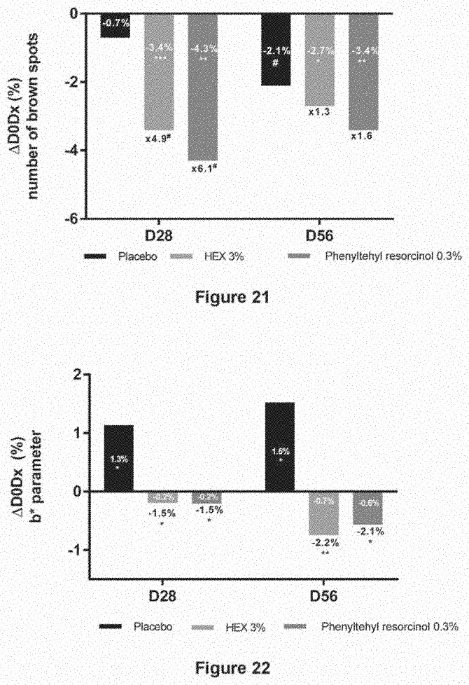

vii) Results: Reduction b* Parameter on Face Pigmented Spots Area (FIG. 22)

A complementary analysis on the colour from pigmented ageing spots area using spectrocolorimeter and especially focusing on b* parameter has been carried out.

The cream containing HEX at 3% significantly reduced the b* parameter which is yellow pigmented that can be correlated to a pheomelanin content (yellow natural pigment). HEX at 3% is also significantly better than placebo showing a significant difference between the both product after 28 and 56 days of application.

Regarding the efficacy of Phenylethyl Resorcinol, the same whitening efficacy as for HEX at 3% has been observed by the significant reduction of b* parameter after 28 and 56 days of application meaning a reduction of yellow colour of the skin. So HEX 3% is as efficient as Phenylethyl resorcinol 0.3% on reduction of b* parameter on pigmented ageing spots on face from Asian volunteers. It was shown, that Himanthalia elongata extract can reduce the b* parameter on pigmented ageing spots on face, in particular of Asian volunteers.

### EXAMPLE 12: CLINICAL TESTING—REDUCTION OF HYPER PIGMENTED SPOTS ON FACE OF AFRICAN VOLUNTEERS

i) INCI Formula Used

The formula described in example 10i) has been used.

ii) Panel Description

The study was a double-blind, randomized and placebo controlled study. The assessment was based on an inter-subject comparison between the results obtained with cream HEX at 3% (active product), cream Phenylethyl resorcinol at 0.3% and the cream placebo. The measurements were taken in controlled-atmosphere at a temperature of 20° C. and a humidity of 40%. This study was performed on 70 volunteers (aged from 19 to 57 years, having hyperpigmentation spots on their faces.

iii) Treatment

The study was carried out on 3 groups. A cream formula was applied twice daily on full face. Evaluation was carried out before treatment and after 28 and 56 days. Groups 1 and 2, with 25 volunteers each, have applied placebo and HEX at 3%, respectively, and group 3 with 20 volunteers has applied Phenylethyl Resorcinol at 0.3%.

iv) Method: Mexameter® Analysis

see Example 10 iv)

v) Results:

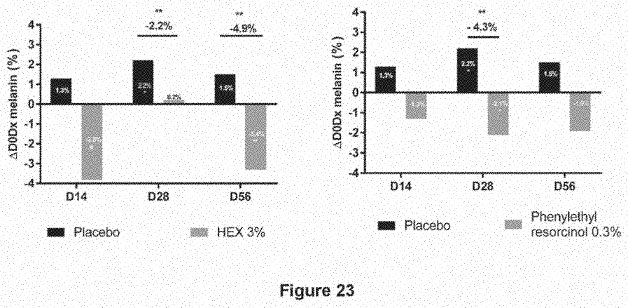

Reduction of Melanin Content (FIG. 23)

HEX at 3% was able to significantly reduce the melanin content after 56 days with −3.4% relative to D0. This effect is also significantly different than the placebo after 56 days of application with −4.9% (FIG. 23 left).

Phenylethyl Resorcinol, used here as a benchmark, caused a slight reduction of melanin content on hyperpigmented area with a significant effect only after 28 days with −2.1% which is not maintained after 56 days. This effect is significantly different than placebo only after 28 days of application with −4.3% (FIG. 23 right). This result evidenced that Phenylethyl resorcinol seems to be less efficient than Himanthalia elongata extract on the reduction of hyper-pigmented spots from African skin.

The efficacy of both products has been compared, and it was observed that there is no relevant difference, demonstrating that Himanthalia elongata extract is as efficient as the benchmark on the reduction of hyper-pigmented spots on African skin related to the comparison with D0 and placebo. It was shown, that Himanthalia elongata extract can reduce hyper-pigmented spots on skin, in particular on African volunteers.

1-14. (canceled)

15. A method for skin treatment comprising administering a Himanthalia elongata extract to skin, wherein the skin treatment comprises at least one of:
(a) skin whitening;
(b) reduction of oxidized proteins after UV exposure;
(c) reduction of lipofuscin bodies accumulation after UV exposure;
(d) reduction of inflammation after UV exposure; and
(e) reduction of Stratum corneum thickness after UV exposure.

16. The method of claim 1, wherein the skin treatment comprises skin whitening.

17. The method of claim 1, wherein the skin treatment comprises reduction of oxidized proteins after UV exposure.

18. The method of claim 1, wherein the skin treatment comprises reduction of lipofuscin bodies accumulation after UV exposure.

19. The method of claim 1, wherein the skin treatment comprises reduction of inflammation after UV exposure.

20. The method of claim 1, wherein the skin treatment comprises reduction of Stratum corneum thickness after UV exposure.

21. The method of claim 1, wherein the Himanthalia elongata extract is applied to the skin in a cosmetic composition.

22. The method of claim 21, wherein the Himanthalia elongata extract is present in an amount of about 2 to about 5% by weight based on the total weight of the cosmetic composition.
